(19) United States
(12) Patent Application Publication
(10) Pub. No.: US 2022/0023359 A1
(43) Pub. Date: Jan. 27, 2022

(54) IDENTIFICATION OF GUT BACTERIA THAT PROMOTE AN ANTI-TUMOR RESPONSE TO IMMUNOTHERAPY

(71) Applicant: President and Fellows of Flarvard College, Cambridge, MA (US)

(72) Inventors: Francesca S. Gazzaniga, Jamaica Plain, MA (US); Dennis Kasper, Brookline, MA (US); Joon Seok Park, Brookline, MA (US); Arlene H. Sharpe, Brookline, MA (US)

(21) Appl. No.: 17/311,587

(22) PCT Filed: Dec. 6, 2019
(86) PCT No.: PCT/US19/65036

§ 371 Date: Jun. 7, 2021

Related U.S. Application Data
(60) Provisional application No. 62/776,767, filed on Dec. 7, 2018.

(51) Int. Cl. A61K 35/742; A61K 39/395; C07K 16/28; A61P 35/00; A23L 33/135; A23L 33/00

(52) U.S. Cl. CPC A61K 35/742; A61K 39/3955; C07K 16/2827; A23V 2002/00; A23L 33/135; A23L 33/40; A61P 35/00

(57) ABSTRACT

In certain aspects, provided herein are methods and compositions for treating or preventing cancer in a subject by conjointly administering to a subject a composition comprising a bacterium (e.g., a bacterium species or strain disclosed herein) and an immunotherapeutic agent.

### RELATED APPLICATIONS

This application claims priority to U.S. Provisional Application 62/776,767, filed Dec. 7, 2018, which is incorporated herein by reference in its entirety.

### BACKGROUND

Immune checkpoint blockade, or immunotherapy, is a novel therapeutic approach that reinvigorates tumor-specific T cells to efficiently kill cancer cells by blocking inhibitory pathways in T cells including CTLA-4 and PD-1. In recent years, antibodies against immune checkpoint molecules have attracted attention as new therapeutic agents for cancer. Immune checkpoint inhibitors promote the activation of T cells by inhibiting a molecule that suppresses the activation and function of T cells, and enhances the antitumor response of the T cells. In a treatment with an immune checkpoint inhibitor, cancer is eliminated by activating the immune state of the living body.

Despite the clinical success of immune checkpoint blockade-based drugs, a significant fraction of cancer patients do not respond to the therapy. Therefore, understanding the elements that regulate the efficacy of the checkpoint blockade is crucial to develop more effective therapies.

### SUMMARY

Provided herein are methods of treating or preventing cancer in a subject or enhancing the efficacy of an immunotherapy in a subject in need thereof. In some aspects, provided herein are methods of treating or preventing cancer in a subject or enhancing the effect of an immunotherapy in a subject in need thereof by conjointly administering to a subject a composition comprising a Clostridia bacterium and an immunotherapeutic agent. At least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the bacteria in the composition may be a Clostridia bacterium.

In some aspects, provided herein are methods of treating or preventing cancer in a subject or enhancing the efficacy of an immunotherapy in a subject in need thereof by conjointly administering to a subject a composition comprising at least one bacteria species selected from Clostridium innocuum, Erysipelatoclostridium ramosum, Coprobacillus catenaformis, Bacteroides dorei, Flavonifractor plautii, Blautia hydrogenotrophica and combinations thereof, and an immunotherapeutic agent. At least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the bacteria in the composition may be at least one bacteria species selected from Clostridium innocuum, Erysipelatoclostridium ramosum, Coprobacillus catenaformis, Bacteroides dorei, Flavonifractor plautii, and Blautia hydrogenotrophica.

In some aspects, provided herein are methods of treating or preventing cancer in a subject or enhancing the efficacy of an immunotherapy in a subject in need thereof by conjointly administering to a subject a composition comprising Erysipelatoclostridium ramosum and an immunotherapeutic agent. At least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the bacteria in the composition may be Erysipelatoclostridium ramosum.

In some aspects, provided herein are methods of treating or preventing cancer in a subject or enhancing the efficacy of an immunotherapy in a subject in need thereof by conjointly administering to a subject a composition comprising Coprobacillus catenaformis and an immunotherapeutic agent. At least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the bacteria in the composition may be Coprobacillus catenaformis.

In some aspects, provided herein are methods of treating or preventing cancer in a subject or enhancing the efficacy of an immunotherapy in a subject in need thereof by conjointly administering to a subject a composition comprising Clostridium innocuum and an immunotherapeutic agent. At least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the bacteria in the composition may be Clostridium innocuum.

In some aspects, provided herein are methods of treating or preventing cancer in a subject or enhancing the efficacy of an immunotherapy in a subject in need thereof by conjointly administering to a subject a composition comprising Flavonifractor plautii and an immunotherapeutic agent. At least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the bacteria in the composition may be Flavonifractor plautii.

In some aspects, provided herein are methods of treating or preventing cancer in a subject or enhancing the efficacy of an immunotherapy in a subject in need thereof by conjointly administering to a subject a composition comprising Blautia hydrogenotrophica and an immunotherapeutic agent. At least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the bacteria in the composition may be Blautia hydrogenotrophica.

In some aspects, provided herein are methods of treating or preventing cancer in a subject or enhancing the efficacy of an immunotherapy in a subject in need thereof by conjointly administering to a subject a composition comprising Clostridium innocuum, Erysipelatoclostridium ramosum, Coprobacillus catenaformis and Bacteroides dorei and an immunotherapeutic agent. At least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the bacteria in the composition may be Clostridium innocuum, Erysipelatoclostridium ramosum, Coprobacillus catenaformis and Bacteroides dorei.

In some aspects, provided herein are methods of treating or preventing cancer in a subject or enhancing the efficacy of an immunotherapy in a subject in need thereof by conjointly administering to a subject a composition comprising Clostridium innocuum, Erysipelatoclostridium ramosum, Coprobacillus catenaformis, Bacteroides dorei, Flavonifractor plautii and Blautia hydrogenotrophica and an immunotherapeutic agent. At least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the bacteria in the composition may be Clostridium innocuum, Erysipelatoclostridium ramosum, Coprobacillus catenaformis, Bacteroides dorei, Flavonifractor plautii and Blautia hydrogenotrophica.

In some embodiments, the composition comprising bacteria further comprises a pharmaceutically acceptable carrier. In some embodiments, the composition is a food product supplemented with the bacteria (e.g., a bacteria disclosed herein). In some embodiments, the food product is or comprises a dairy product (e.g., yogurt, frozen yogurt, ice cream, milk or cheese). In some embodiments, the food product is a non-dairy food product. In some embodiments, the food product is a beverage. In some embodiments, the composition is administered orally. In some embodiments, the composition is administered rectally.

In some embodiments, the immunogenic composition comprises an immune checkpoint inhibitor. The immune checkpoint inhibitor may comprise an immune checkpoint inhibitor comprises an antibody specific for an immune checkpoint protein selected from CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, PVRIG, (CD112R), HHLA2, butyrophilins, and A2aR. In some embodiments, the immune checkpoint inhibitor is cemiplimab, nivolumab, pembrolizumab, atezolizumab, durvalumab, avelumab, or ipilimumab.

In some embodiments of the methods described herein, the subject has reduced levels of bacteria present in their gut. In some embodiments, the subject had been administered an antibiotic. In some embodiments, the antibiotic was administered less than a month, less than 30, 28, 21, 14, 13, 12, 11, 10, 9, 8, 7, 6, 5, 4, 3, 2 or 1 days prior to administration of the composition of the present invention.

In some embodiments, the cancer is lung cancer, breast cancer, colon cancer, pancreatic cancer, renal cancer, stomach cancer, a GI cancer, liver cancer, bone cancer, hematological cancer, neural tissue cancer, melanoma, thyroid cancer, ovarian cancer, testicular cancer, prostate cancer, cervical cancer, vaginal cancer, or bladder cancer.

In some embodiments, the cancer comprises a solid tumor. The tumor may be an adenocarcinoma, an adrenal tumor, an anal tumor, a bile duct tumor, a bladder tumor, a bone tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngeal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor. The tumor may be a primary tumor or a metastatic tumor.

In some embodiments, the subject has received a chemotherapy drug prior to administration of the agent. The subject may re refractory to a chemotherapy drug. The immunogenic composition may be administered by any means known in the art, including systemically or intravenously. The immunogenic composition may be administered subcutaneously, intramuscularly, or locally (e.g., to the location of the cancer cells, such as the tumor or tumor microenvironment).

In certain aspects, provided herein is a method of making a composition for enhancing the efficacy of a cancer immunotherapy in a subject by combining at least one bacteria disclosed herein with a pharmaceutically acceptable carrier. In some embodiments, the composition is formulated for oral administration. In some embodiments, the composition is formulated for rectal administration.

In certain aspects, provided herein is a method of making a composition for enhancing the efficacy of a cancer immunotherapy in a subject by combining at least one bacteria disclosed herein with a food product. In some embodiments, the food product is or comprises a dairy product (e.g., yogurt, frozen yogurt, ice cream, milk or cheese). In some embodiments, the food product is a non-dairy food product. In some embodiments, the food product is a beverage.

In some embodiments, the bacteria are live, replication competent bacteria.

In some embodiments of the compositions and methods described herein, at least 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition are selected from the group consisting of Clostridium innocuum, Erysipelatoclostridium ramosum, Coprobacillus catenaformis, Bacteroides dorei, Flavonifractor plautii, and Blautia hydrogenotrophica.

### BRIEF DESCRIPTION OF THE DRAWINGS

FIG. 1 has three parts, A-C, shows gut microbiota is essential for immune checkpoint blockade mediated anti-tumor immunity. 2.5×10⁵ of MC38 cells were implanted subcutaneously in GF (germ-free) mice (Part A) and Hmb-colonized mice (Part B). Hmb is human microbiota isolated from germ free mice that have been colonized with healthy human feces and have been maintained in isolators for 20+ generations. Antibiotics-treated mice were colonized with Hmb by oral gavage a week after tumor implantation (Part C). 100 μg of anti-PD-L1 antibody (10F.9G2) or isotype control (IgG2b) was intraperitoneally injected to the tumor bearing mice on post-implantation day 7, 10, 13 and 16. The broad-spectrum antibiotics cocktail containing 0.5 mg/ml of vancomycin, 1 mg/ml of neomycin, 1 mg/ml of ampicillin and 1 mg/ml of metronidazole was administered into mice through drinking water, starting four days before tumor implantation and continued throughout the experiment in the Broad Spectrum Antibiotics group but was stopped in mice colonized with Hmb at day 7. Tumor size was measured every three days from post-implantation day 7.

FIG. 2 shows gram-positive anaerobic bacteria are responsible for anti-tumor immunity upon anti-PD-L1 treatment. C57BL/6 mice (Taconic farm) were treated with the broad-spectrum antibiotics from day −4 to day 7. On day 7, Hmb was given to mice, followed by treatment of individual antibiotics as indicated. Tumor experiments were performed as described. Arrows indicate death of mice with large tumors in metronidazole and vancomycin treated mice.

FIG. 3 has three parts, A-C, and shows Clostridiales in human microbiota is responsible for anti-tumor immunity. Germ-free mice were colonized with a mix of 6 Clostridiales and one Bacteroides species (Part A), a mix of 4 Clostridiales and Bacteroides species (Clostridium innocuum, Erysipelatoclostridium ramosum, Coprobacillus catenaformis and Bacteroides dorei) (Part B) and Hmb #7 (Clostridium innocuum) isolate (Part C) a week before tumor implantation. Tumor experiments were performed as described.

FIG. 4 shows anti-tumor responses to anti-PD-L1 immunotherapy depends on gut microbiota. This figure shows mice treated with antibiotics as described do not respond to anti-PD-L1 therapy, but mice treated with antibiotics and orally gavaged with Hmb 7 days after tumor implantation do respond to anti-PD-L1 immunotherapy.

FIG. 5 shows anti-tumor responses to anti-PD-L1 immunotherapy depends on specific gut microbiota. Part A shows germ free mice do not respond to anti-PD-L1 therapy, but germ free mice colonized with Hmb 7 days prior to tumor implantation (Part B) do respond to anti-PD-L1 therapy and mice colonized with Bacillus circulans (Part C) do not respond to anti-PD-L1 therapy.

FIG. 6 shows Clostridium innocuum (Hmb #7) increases the efficacy of anti-PD-L1 therapy by 40-60%.

FIG. 7 shows that in mice treated with antibiotics four days before tumor implantation until 7 days after tumor implantation, live bacteria daily therapy starting at day 7 administered conjointly with an immune checkpoint inhibitor increases tumor killing compared to mice that were treated with antibiotics throughout the study and given immune checkpoint inhibitor. (Clostridium innocuum (Top) and live Erysipelatoclostridium ramosum (Bottom)).

FIG. 8 shows that in mice treated with antibiotics four days before tumor implantation until 7 days after tumor implantation, killed bacteria daily therapy starting at day 7 administered conjointly with an immune checkpoint inhibitor increases tumor killing compared to mice that were treated with antibiotics throughout the study and given immune checkpoint inhibitor (Bacteroides dorei (Top) and killed Coprobacillus catenaformis (Bottom)).

FIG. 9 has two parts, A-B, and shows Coprobacillus cateniformis and Erysipelatoclostridium ramosum promotes anti-tumor immunity upon PD-L1 blockade in germ-free mice. Germ-free mice were mono-colonized with Coprobacillus cateniformis (Part A) or Erysipelatoclostridium ramosum (Part B) isolated from human microbiota a week before MC38 tumor implantation. The mice were treated with anti-PD-L1 antibody (10F.9G2), and monitored for tumor growth.

### DETAILED DESCRIPTION

### General

In certain aspects, provided herein are methods and compositions for treating or preventing cancer in a subject by conjointly administering to a subject a composition comprising a bacterium (e.g., a bacterium species or strain disclosed herein) and an immunotherapeutic agent. In some embodiments, the compositions provided herein enhance the anti-tumor activity of the immunotherapeutic agent.

The composition may comprise a Clostridum bacterium. The composition may comprise a Bacteroides bacterium. The composition may comprise at least one bacteria species selected from Clostridium innocuum, Erysipelatoclostridium ramosum, Coprobacillus catenaformis, Bacteroides dorei, Flavonifractor plautii, Blautia hydrogenotrophica or combinations thereof. In some embodiments, the composition comprises at least one (e.g., at least two, at least three, at least four, or at least five) species of bacteria selected from Clostridium innocuum, Erysipelatoclostridium ramosum, Coprobacillus catenaformis, Bacteroides dorei, Flavonifractor plautii, or Blautia hydrogenotrophica, and the bacteria enhances the anti-tumor activity of the immunotherapeutic agent.

### Definitions

For convenience, certain terms employed in the specification, examples, and appended claims are collected here.

The articles “a” and “an” are used herein to refer to one or to more than one (i.e., to at least one) of the grammatical object of the article.

As used herein, the term “administering” means providing an agent or composition to a subject, and includes, but is not limited to, administering by a medical professional and self-administering.

As used herein, an “effective amount” is an amount effective in treating or preventing a disease associated with a pathological immune response, including, for example, inflammatory bowel disease.

The terms “prevent,” “preventing,” “prevention,” and the like refer to reducing the probability of developing a disease, disorder, or condition in a subject, who does not have, but is at risk of or susceptible to developing a disease, disorder, or condition.

As used herein, the term “subject” means a human or non-human animal selected for treatment or therapy. In certain embodiments, of the methods and compositions described herein the subject is a human subject.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

### Bacteria

In certain aspects, provided herein are methods and compositions for treating or preventing cancer in a subject by conjointly administering to a subject a composition comprising a bacterium (e.g., a bacterium species or strain disclosed herein) and an immunotherapeutic agent. In some embodiments, the compositions provided herein enhance the anti-tumor activity of the immunotherapeutic agent. In certain aspects, provided herein are methods and compositions for enhancing, increasing or potentiating the effect of a cancer immunotherapy (e.g., an immune checkpoint inhibitor) in a subject in need thereof by conjointly administering to a subject a composition comprising a bacterium (e.g., a bacterium species or strain disclosed herein) and an immunotherapeutic agent. The composition may comprise a Clostridum bacterium. The composition may comprise a Bacteroides bacterium. The composition may comprise at least one (e.g., at least two, at least three, at least four, at least five, or all six) bacteria species selected from Clostridium innocuum, Erysipelatoclostridium ramosum, Coprobacillus catenaformis, Bacteroides dorei, Flavonifractor plautii, and Blautia hydrogenotrophica.

In some embodiments, the bacteria described herein have a genomic sequence that comprises at least 90%, 91%, 92%, 93%, 94%, 95%, 96%, 97%, 98%, 99% or 100% homology to the genomic sequence of Clostridium innocuum, Erysipelatoclostridium ramosum, Coprobacillus catenaformis, Bacteroides dorei, Flavonifractor plautii, or Blautia hydrogenotrophica. In some embodiments, the bacteria described herein has a 16s ribosomal sequence that comprises at least 90%, 91%, 92%, 93%, 94%, 95%, 96%, 97%, 98%, 99% or 100% homology to the 16s ribosomal sequence of Clostridium innocuum, Erysipelatoclostridium ramosum, Coprobacillus catenaformis, Bacteroides dorei, Flavonifractor plautii, or Blautia hydrogenotrophica. The bacteria described herein can be grown in culture using methods known in the art. For example, the bacteria can be grown in supplemented Yeast extract-peptone-glycerol (YPG) medium, chopped meat broth, Blood Brucella Agar or Blood TSA Agar plates.

In some embodiments, combinations of strains of bacteria (e.g., bacteria disclosed herein) are used in the methods and/or compositions provided herein. In certain embodiments, a combination of at least 2, 3, 4, 5, 6, 7, 8, 9, 10, 11, 12, 13, 14, 15, 16, 17, 18 or 19 of strains of bacteria are used in the methods and/or compositions described herein. A composition disclosed herein may comprise a strain or several strains from one or more species of bacteria disclosed herein.

In some embodiments, the compositions disclosed herein comprise a combination of bacterial strain or species disclosed herein.

In some embodiments, the combination of bacteria includes Erysipelatoclostridium ramosum and one or more bacterial species or strains selected from the group consisting of Clostridium innocuum, Coprobacillus catenaformis, Bacteroides dorei, Flavonifractor plautii, and Blautia hydrogenotrophica.

In some embodiments, the combination of bacteria includes Coprobacillus catenaformis, and one or more bacterial species or strains selected from the group consisting of Erysipelatoclostridium ramosum, Clostridium innocuum, Bacteroides dorei, Flavonifractor plautii, and Blautia hydrogenotrophica.

In some embodiments, the combination of bacteria includes Clostridium innocuum and one or more bacterial species or strains selected from the group consisting of Coprobacillus catenaformis, Erysipelatoclostridium ramosum, Bacteroides dorei, Flavonifractor plautii and Blautia hydrogenotrophica.

In some embodiments, the combination of bacteria includes Bacteroides dorei and one or more bacterial species or strains selected from the group consisting of Coprobacillus catenaformis, Clostridium innocuum, Erysipelatoclostridium ramosum, Flavonifractor plautii, and Blautia hydrogenotrophica.

In some embodiments, the combination of bacteria includes Flavonifractor plautii and one or more bacterial species or strains selected from the group consisting of Coprobacillus catenaformis, Clostridium innocuum, Erysipelatoclostridium ramosum, Bacteroides dorei and Blautia hydrogenotrophica.

In some embodiments, the combination of bacteria includes Blautia hydrogenotrophica and one or more bacterial species or strains selected from the group consisting of Coprobacillus catenaformis, Clostridium innocuum, Erysipelatoclostridium ramosum, Bacteroides dorei and Flavonifractor plautii.

In some embodiments, the combination of bacteria includes Erysipelatoclostridium ramosum and Clostridium innocuum. In some embodiments, the combination of bacteria includes Erysipelatoclostridium ramosum and Coprobacillus catenaformis. In some embodiments, the combination of bacteria includes Erysipelatoclostridium ramosum and Bacteroides dorei. In some embodiments, the combination of bacteria includes Erysipelatoclostridium ramosum and Flavonifractor plautii. In some embodiments, the combination of bacteria includes Erysipelatoclostridium ramosum and Blautia hydrogenotrophica.

In some embodiments, the combination of bacteria includes Clostridium innocuum and Coprobacillus catenaformis. In some embodiments, the combination of bacteria includes Clostridium innocuum and Erysipelatoclostridium ramosum. In some embodiments, the combination of bacteria includes Clostridium innocuum and Bacteroides dorei. In some embodiments, the combination of bacteria includes Clostridium innocuum and Flavonifractor plautii. In some embodiments, the combination of bacteria includes Clostridium innocuum and Blautia hydrogenotrophica.

In some embodiments, the combination of bacteria includes Coprobacillus catenaformis and Clostridium innocuum. In some embodiments, the combination of bacteria includes Coprobacillus catenaformis and Erysipelatoclostridium ramosum. In some embodiments, the combination of bacteria includes Coprobacillus catenaformis and Bacteroides dorei. In some embodiments, the combination of bacteria includes Coprobacillus catenaformis and Flavonifractor plautii. In some embodiments, the combination of bacteria includes Coprobacillus catenaformis and Blautia hydrogenotrophica.

In some embodiments, the combination of bacteria includes Bacteroides dorei and Coprobacillus catenaformis. In some embodiments, the combination of bacteria includes Bacteroides dorei and Clostridium innocuum. In some embodiments, the combination of bacteria includes Bacteroides dorei and Erysipelatoclostridium ramosum. In some embodiments, the combination of bacteria includes Bacteroides dorei and Flavonifractor plautii. In some embodiments, the combination of bacteria includes Bacteroides dorei and Blautia hydrogenotrophica.

In some embodiments, the combination of bacteria includes Flavonifractor plautii, and Coprobacillus catenaformis. In some embodiments, the combination of bacteria includes Flavonifractor plautii, and Clostridium innocuum. In some embodiments, the combination of bacteria includes Flavonifractor plautii, and Erysipelatoclostridium ramosum. In some embodiments, the combination of bacteria includes Flavonifractor plautii, and Bacteroides dorei. In some embodiments, the combination of bacteria includes Flavonifractor plautii, and and Blautia hydrogenotrophica.

In some embodiments, the combination of bacteria includes Blautia hydrogenotrophica and Coprobacillus catenaformis. In some embodiments, the combination of bacteria includes Blautia hydrogenotrophica and Clostridium innocuum. In some embodiments, the combination of bacteria includes Blautia hydrogenotrophica and Erysipelatoclostridium ramosum. In some embodiments, the combination of bacteria includes Blautia hydrogenotrophica and Bacteroides dorei. In some embodiments, the combination of bacteria includes Blautia hydrogenotrophica and Flavonifractor plautii. In certain aspects, provided herein are methods and compositions for treating or preventing cancer in a subject by conjointly administering to a subject a composition comprising Clostridium innocuum, Erysipelatoclostridium ramosum, Coprobacillus catenaformis and Bacteroides dorei and an immunotherapeutic agent. In some embodiments, the compositions provided herein enhance the anti-tumor activity of the immunotherapeutic agent.

### Compositions

In certain embodiments, provided herein is a composition (e.g., a pharmaceutical composition, a dietary supplement or a food product) containing bacteria or combinations of bacteria that enhance the performance of an immune checkpoint inhibitor. In some embodiments, the composition further comprises a pharmaceutically acceptable carrier.

In some embodiments, at least 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition are selected from among the bacterial species described herein. 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition are selected from among the bacterial strains described herein.

In some embodiments, the compositions described herein may include only one strain of the bacteria described herein or may include two or more strains of the bacteria described herein. For example, 1, 2, 3, 4, 5, 6, or 7 of the species and/or strains described herein, in any combination, can be included in the compositions provided herein.

In some embodiments, the composition comprising bacteria (e.g., a bacteria disclosed herein) described herein may be a pharmaceutical composition, a dietary supplement, or a food product (e.g., a food or beverage). In some embodiments, the food product is an animal feed.

As described in detail below, the pharmaceutical compositions disclosed herein may be specially formulated for administration in solid or liquid form, including those adapted for oral or rectal administration.

In certain embodiments, the pharmaceutical composition comprising a bacteria disclosed herein for oral administration comprises an additional component that enables efficient delivery of the bacteria to the colon. In some embodiments, pharmaceutical preparation that enables the delivery of the bacteria to the colon can be used. Examples of such formulations include pH sensitive compositions, such as buffered sachet formulations or enteric polymers that release their contents when the pH becomes alkaline after the enteric polymers pass through the stomach. When a pH sensitive composition is used for formulating the pharmaceutical preparation, the pH sensitive composition can be a polymer whose pH threshold of the decomposition of the composition is between about 6.8 and about 7.5.

Another embodiment of a pharmaceutical composition useful for delivery of the bacteria to the colon is one that ensures the delivery to the colon by delaying the release of the bacteria by days to hours, including approximately 3 to 5 hours, which corresponds to the small intestinal transit time. In some embodiments, the pharmaceutical composition for delayed release includes a hydrogel shell. The hydrogel is hydrated and swells upon contact with gastrointestinal fluid, with the result that the contents are effectively released (released predominantly in the colon). Delayed release dosage units include bacteria-containing compositions having a material which coats or selectively coats the bacteria. Examples of such a selective coating material include in vivo degradable polymers, gradually hydrolyzable polymers, gradually water-soluble polymers, and/or enzyme degradable polymers. A wide variety of coating materials for efficiently delaying the release is available and includes, for example, cellulose-based polymers such as hydroxypropyl cellulose, acrylic acid polymers and copolymers such as methacrylic acid polymers and copolymers, and vinyl polymers and copolymers such as polyvinylpyrrolidone.

Examples of compositions enabling the delivery to the colon further include bioadhesive compositions which specifically adhere to the colonic mucosal membrane (for example, a polymer described in the specification of U.S. Pat. No. 6,368,586, hereby incorporated by reference) and compositions into which a protease inhibitor is incorporated for protecting particularly a biopharmaceutical preparation in the gastrointestinal tracts from decomposition due to an activity of a protease.

An example of a system enabling the delivery to the colon is a system of delivering a composition to the colon by pressure change in such a way that the contents are released by utilizing pressure change caused by generation of gas in bacterial fermentation at a distal portion of the stomach. Such a system is not particularly limited, and a more specific example thereof is a capsule which has contents dispersed in a suppository base and which is coated with a hydrophobic polymer (for example, ethyl cellulose).

Another example of the system enabling the delivery to the colon is a system of delivering a composition to the colon, the system being specifically decomposed by an enzyme (for example, a carbohydrate hydrolase or a carbohydrate reductase) present in the colon. Such a system is not particularly limited, and more specific examples thereof include systems which use food components such as non-starch polysaccharides, amylose, xanthan gum, and azopolymers.

In some embodiments, the composition is a food product (e.g., a food or beverage) such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Specific examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, soups; dairy products such as milk, dairy beverages, ice creams, cheeses, and yogurts; fermented products such as fermented soybean pastes, yogurts, fermented beverages, and pickles; bean products; various confectionery products, including biscuits, cookies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

In some embodiments the composition is a food product for animals, including humans.

### Methods

In some aspects, provided herein are methods of treating or preventing cancer in a subject or enhancing the effect of an immunotherapy in a subject in need thereof by conjointly administering to a subject a bacterium (e.g., a bacterium species or strain disclosed herein) and an immunotherapeutic agent. The methods described herein can be used to treat any subject in need thereof. As used herein, a “subject in need thereof” includes any subject that has cancer, as well as any subject with an increased likelihood of acquiring cancer.

The methods provided herein include administering an additional anti-cancer therapy to the subject. The additional anti-cancer therapy may be a cancer immunotherapy, such as an autologous or allogeneic T-cell therapy, an autologous or allogenic CAR T-cell therapy, administering TNF-α, or administering an immune checkpoint inhibitor to the subject. The immune checkpoint inhibitor may comprise an antibody specific for an immune checkpoint protein selected from CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, PVRIG, (CD112R), HHLA2, butyrophilins, and A2aR. The immune checkpoint protein may be a bi-specific antibody. In some embodiments, the immune checkpoint inhibitor is cemiplimab (REGN2810), nivolumab (BMS-936558, MDX-1106, ONO-4538), pembrolizumab (MK-3475, SCH 900475), atezolizumab (MPDL3280A, RG7446, R05541267), durvalumab (MEDI4736, MEDI-4736), avelumab (MSB0010718C), ipilimumab (BMS-734016, IBI310, MDX-010), SHR1210, sintilimab (IBI308), spartalizumab (PDR001), tislelizumab (BGB-A317), pidilizumab, BCD-100, toripalimab (JS001), BAY 1905254, ASP 8374, PF-06801591, AMP-224, AB122, AK105, AMG 404, BCD-100, BI 754091, F520, HLX10, HX008, JTX-4014, LZMO09, MEDI0680, MGA012, Sym021, TSR-042, PSB205, MGD019, MGD013, AK104, XmAb20717, R07121661, CX-188, INCB086550, FS118, BCD-135, BGB-A333, CBT-502, CK-301, CS1001, FAZ053, HLX20, KN035, MDX-1105, MSB2311, SHR-1316, TG-1501, ZKAB001, INBRX-105, MCLA-145, KN046, M7824, LY3415244, INCB086550, CA-170, CX-072, ADU-1604, AGEN1181, AGEN1884, MK-1308, REGN4659, XmAb22841, ATOR-1015, PSB205, MGD019, AK104, XmAb20717, BMS-986249, tremelimumab, BMS-986258, BGB-A425, INCAGN02390, Sym023, JNJ 61610588, BI 754111, LAG525, MK-4280, REGN3767, Sym022, TSR-033, relatlimab, JTX-2011, MGD009, BMS-986207, OMP-313M32, MK-7684 or TSR-022. In some embodiments, the cancer immunotherapy comprises administering a cancer vaccine to the subject. Additional immune checkpoint inhibitors and details regarding inhibitors can be found in Darvin, P., Toor, S. M., Sasidharan Nair, V. et al. Immune checkpoint inhibitors: recent progress and potential biomarkers. Exp Mol Med 50, 165 (2018) doi:10.1038/s12276-018-0191-1; which is hereby incorporated by reference in its entirety.

In certain embodiments, compositions and agents of the invention may be used alone or conjointly administered with another type of therapeutic agent. As used herein, the phrase “conjoint administration” or “administered conjointly” refers to any form of administration of two or more different therapeutic agents such that the second agent is administered while the previously administered therapeutic agent is still effective in the body (e.g., the two agents are simultaneously effective in the subject, which may include synergistic effects of the two agents). For example, the different therapeutic agents can be administered either in the same formulation or in separate formulations, either concomitantly or sequentially. In certain embodiments, the different therapeutic agents can be administered within about one hour, about 12 hours, about 24 hours, about 36 hours, about 48 hours, about 72 hours, or about a week of one another. Thus, a subject who receives such treatment can benefit from a combined effect of different therapeutic agents.

In some embodiments, the subject has received a chemotherapy drug prior to administration of the agent. The subject may be refractory to a chemotherapy drug. The subject may receive a chemotherapeutic agent sequentially or simultaneously to receiving an agent of additional cancer therapy disclosed herein. Chemotherapeutic agents include alkylating agents such as thiotepa and cyclophosphamide (Cytoxan™); alkyl sulfonates such as busulfan, improsulfan and piposulfan; aziridines such as benzodopa, carboquone, meturedopa, and uredopa; emylerumines and memylamelamines including alfretamine, triemylenemelamine, triethylenephosphoramide, triethylenethiophosphoramide, and trimemylolomelamine; acetogenins (especially bullatacin and bullatacinone); a camptothecin (including synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, foremustine, lomustine, nimustine, ranimustine; antibiotics such as the enediyne antibiotics (e.g., calicheamicin, especially calicheamicin gammall and calicheamicin phili); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (Adramycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as demopterin, methotrexate, pteropterin, trimetrexate; purine analogs such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replinisher such as frolinic acid; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK™; razoxane; rhizoxin; sizofiran; spirogermanium; tenuazonic acid; triaziquone; 2,2′,2″-tricUorotriemylamine; trichothecenes (especially T-2 toxin, verracurin A, roridin A and anguidine); urethane; vindesine; dacarbazine; mannomustine; mitobronitol; mitolactol; pipobroman; gacytosine; arabinoside (“Ara-C”); cyclophosphamide; thiopeta; taxoids, e.g., paclitaxel (Taxol™, Bristol Meyers Squibb Oncology, Princeton, N.J.) and docetaxel (Taxoteret™, Rhone-Poulenc Rorer, Antony, France); chlorambucil; gemcitabine (Gemzar™); 6-thioguanine; mercaptopurine; methotrexate; platinum analogs such as cisplatin and carboplatin; vinblastine; platinum; etoposide (VP-16); ifosfamide; mitroxantrone; vancristine; vinorelbine (Navelbine™); novantrone; teniposide; edatrexate; daunomycin; aminopterin; xeoloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included in the definition of “chemotherapeutic agent” are anti-hormonal agents that act to regulate or inhibit hormone action on tumors such as anti-estrogens and selective estrogen receptor modulators (SERMs), including, for example, tamoxifen (including Nolvadex™) raloxifene, droloxifene, 4-hydroxytamoxifen, trioxifene, keoxifene, LY117018, onapristone, and toremifene (Fareston™); inhibitors of the enzyme aromatase, which regulates estrogen production in the adrenal glands, such as, for example, 4(5)-imidazoles, aminoglutethimide, megestrol acetate (Megace™), exemestane, formestane, fadrozole, vorozole (Rivisor™), letrozole (Femara™), and anastrozole (Arimidex™); and anti-androgens such as flutamide, nilutamide, bicalutamide, leuprohde, and goserelin; and pharmaceutically acceptable salts, acids or derivatives of any of the above.

In some embodiments, administration is in combination with administration of at least one prebiotic substance (e.g., a prebiotic substance that favors the growth of the bacterial species in the composition over the growth of other human commensal bacterial species). In some embodiments, the prebiotic substance is a nondigestible oligosaccharide. In some embodiments, the prebiotic substance is almond skin, inulin, oligofructose, raffinose, lactulose, pectin, hemicellulose, amylopectin, acetyl-Co A, biotin, beet molasses, yeast extracts, and resistant starch.

In some embodiments, the methods provided herein include the step of administering at least one antibiotic before or in combination with, the administration of a composition described herein.

In some embodiments, the methods provided herein include the step of determining the subject's microbiome prior to the administration of a composition described herein. In some embodiments, the selection of the bacteria or combination of bacteria administered to the subject is determined based upon the make-up of the subject's microbiome.

As described in detail below, the pharmaceutical compositions and/or agents disclosed herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous, intrathecal, intracerebral or epidural injection as, for example, a sterile solution or suspension, or sustained-release formulation. Methods of preparing pharmaceutical formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, dimethyl sulfoxide (DMSO), polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Actual dosage levels of the active ingredients in the pharmaceutical compositions or agents to be administered may be varied so as to obtain an amount of the active ingredient (e.g., an agent described herein) which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician having ordinary skill in the art can readily determine and prescribe the effective amount of the pharmaceutical composition required. For example, the physician could prescribe and/or administer doses of the compounds employed in the pharmaceutical composition at levels lower than that required in order to achieve the desired therapeutic effect and gradually increase the dosage until the desired effect is achieved.

### Indications

In some embodiments, the methods described herein may be used to treat any cancer, including any cancerous or pre-cancerous tumor. Cancers that may be treated by methods and compositions provided herein include, but are not limited to, cancer of the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; bronchioloalveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometrioid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; mammary paget's disease; acinar cell carcinoma; adenosquamous carcinoma; adenocarcinoma w/squamous metaplasia; malignant thymoma; malignant ovarian stromal tumor; malignant thecoma; malignant granulosa cell tumor; and malignant roblastoma; sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; small lymphocytic malignant lymphoma; diffuse large cell malignant lymphoma; follicular malignant lymphoma; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancer comprises a solid tumor. In some embodiments, the tumor is an adenocarcinoma, an adrenal tumor, an anal tumor, a bile duct tumor, a bladder tumor, a bone tumor, a blood born tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngeal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor.

### EXEMPLIFICATION

The human gut is colonized by 100 trillion bacteria collectively referred to as the gut microbiota, which plays an essential role in health and disease. Disruption of a healthy gut microbiota, called dysbiosis, is associated with a wide variety of conditions including obesity, colitis, cancer, irritable bowel syndrome, type 1 diabetes, arthritis, allergies, multiple sclerosis, Parkinson's, and autism.

Immune checkpoint blockade, or immunotherapy, is a novel therapeutic approach that reinvigorates tumor-specific T cells to efficiently kill cancer cells by blocking inhibitory pathways in T cells including CTLA-4 and PD-1. Despite the clinical success of immune checkpoint blockade-based drugs, a significant fraction of cancer patients do not respond to the therapy. Therefore, understanding the elements that regulate the efficacy of the checkpoint blockade is crucial to develop more effective therapies.

Gut microbiota between responders and non-responders of immunotherapy may be different, and particular microbes seem to be associated with anti-tumor immunity. Using mice colonized with human bacteria and metagenomic tools, Applicant has successfully identified a mixture of six Clostridiales and one Bacteroides bacteria that in different combinations, and individually, can promote anti-tumor immunity upon αPD-L1 antibody treatment. These bacterial strains could be used individually or in combination as a probiotic that promotes anti-tumor response to immunotherapy.

### Microbiota-Dependent Anti-Tumor Immunity Upon αPD-L1 Treatment

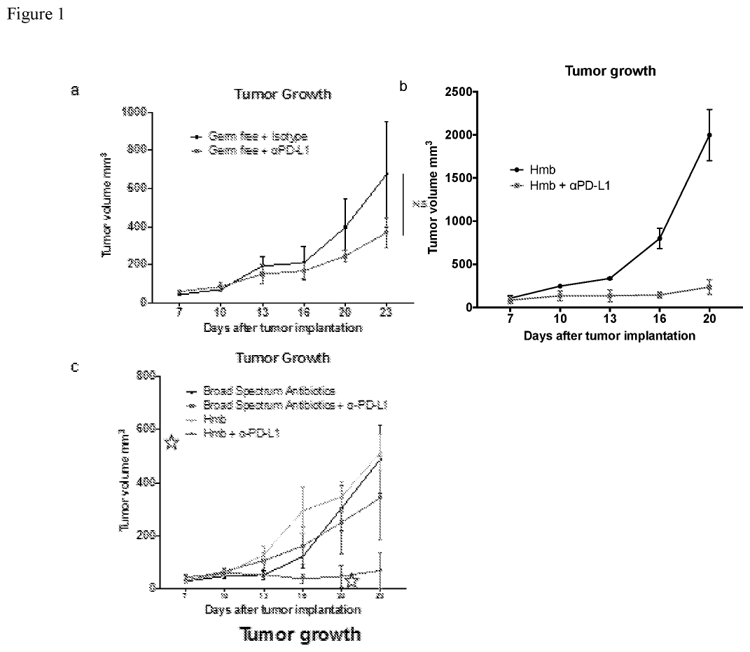

To establish mouse models to study microbiota-dependent anti-tumor immunity, tumors were implanted in germ-free (GF) and antibiotic treated mice. GF mice are housed in sterile isolators and are free of all microbes. Antibiotic treated mice are given a mix of 4 antibiotics that kill a broad spectrum of bacteria to significantly reduce bacterial load and diversity. Consistent with previous reports, αPD-L1 treatment was ineffective in treating or clearing tumors in both GF and antibiotic treated mice (FIG. 1 A, C). The efficacy of αPD-L1 treatment was restored when GF mice were colonized with healthy human microbiota (FIG. 1B) 7 days before tumor implantation or 7 days after implantation for antibiotics-treated mice (FIG. 1C). These results show that the αPD-L1 anti-tumor response is dependent on the microbiota. The human microbiota used in all of the experiments, referred to as Hmb, was isolated from germ free mice that have been colonized with healthy human feces and have been maintained in isolators for 20+ generations.

### Gram-Positive Anaerobic Bacteria are Essential for the Efficacy of αPD-L1 Treatment

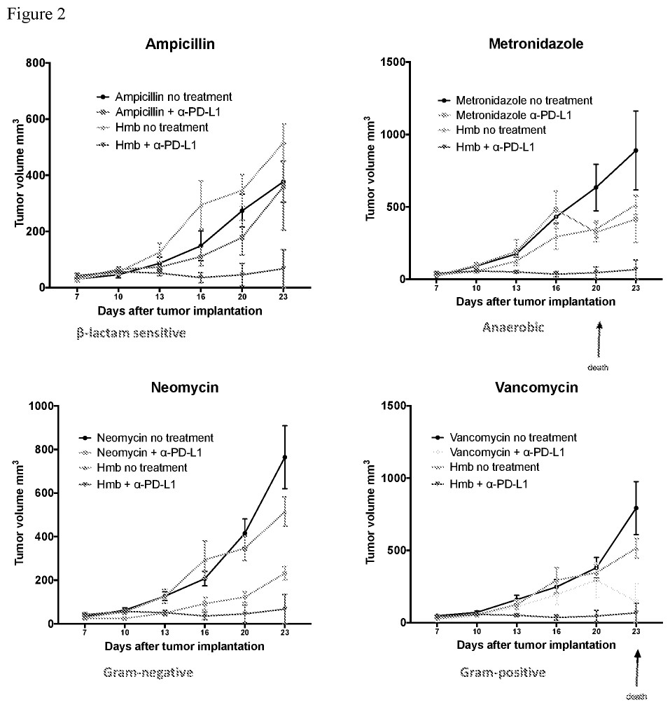

To narrow down the types of microbes that are responsible for the effective anti-tumor immunity, mice were inoculated with Hmb and were treated with single antibiotics that target different categories of bacteria. Whereas administration of neomycin, which targets gram-negative bacteria, did not have a significant impact on the efficacy of αPD-L1 treatment (FIG. 2 left), Metronidazole, Ampicillin, and Vancomycin, which inhibit anaerobic, β-lactam sensitive, and gram-positive bacteria, completely compromised the efficacy (FIG. 2, right). These results indicate that the human gut microbes that are required for anti-tumor immunity upon αPD-L1 are gram-positive anaerobic bacteria.

Clostridiales in Human Microbiota Contains the Bacterial Species that Generate the Effective Anti-Tumor Immunity Upon αPD-L1 Treatment.

For specific identification of the gut microbes promoting anti-tumor immunity, the microbiota of responder and non-responder mice were sequenced. Although more than half of Hmb-colonized mice cleared tumors after αPD-L1 treatment, some mice, did not respond to αPD-L1 treatment. Therefore, 16S sequence analysis of stool samples from responders and non-responders was compared to identify the unique bacterial species that are associated with the responsiveness to the therapy. The comparative analysis showed that there was a significant difference in OTUs of Clostridiales order between responders and non-responders (t-test, p=0.0396). To identify the specific strains of Clostridiales in the Hmb samples, the Hmb stock was anaerobically cultured on brain heart infusion plates, which lack hemin and vitamin k, and thus enrich for Clostridiales species. Individual colonies were isolated and identified six different Clostridiales species and one Bacteroides in the human microbiota samples (Table 1). For some strains, a putative species has been listed based on 16s sequencing, but further biochemical testing and sequencing is being completed to confirm the identity of these strains.

TABLE 1

Hmb #1 Unknown gram positive rod shaped bacterium
Hmb #2 Blautia hydrogenotrophica
Hmb #4 Flavonifractor plautii/Clostridia orbiscindens
Hmb #7 Clostridium innocuum
Hmb # 11 Bacteroides dorei
Hmb #21 Coprobacillus cateniformis/Candidatus stoquefichus
Hmb #24 Erysipelatoclostrdidium ramosum

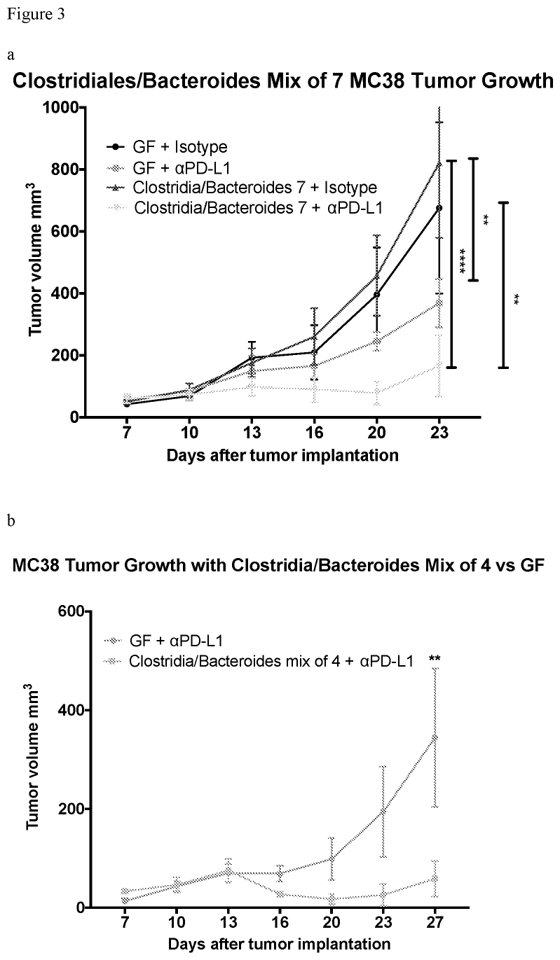

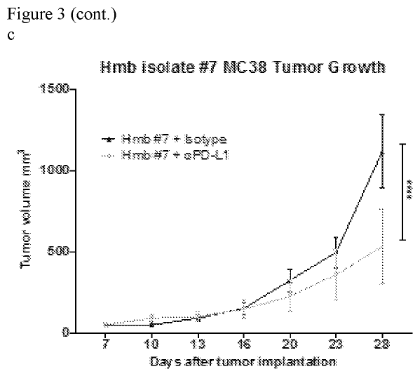

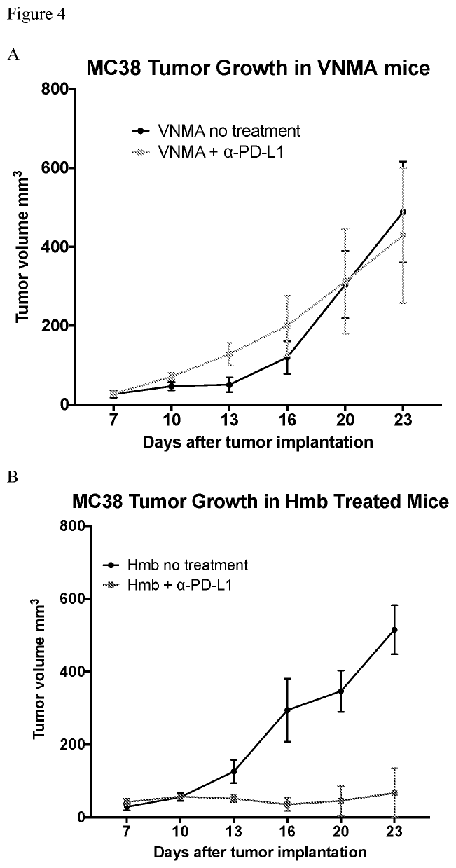

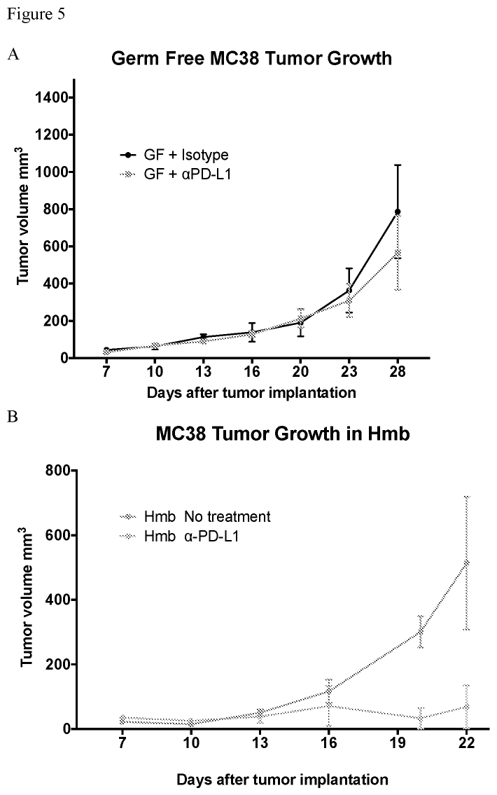

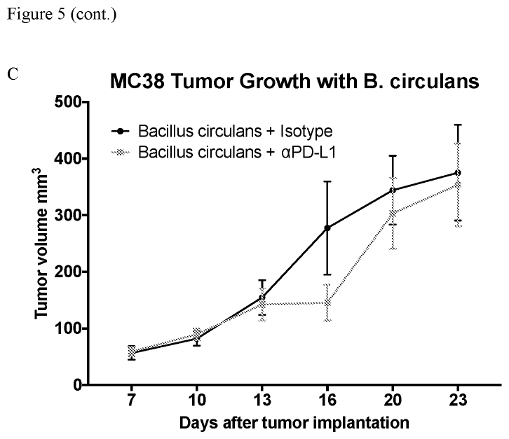

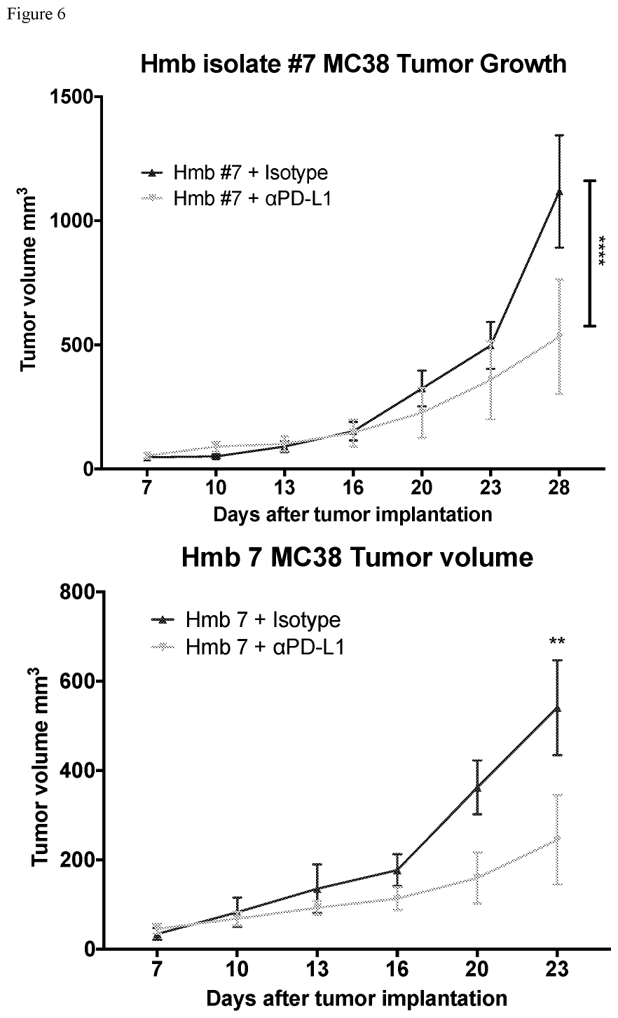

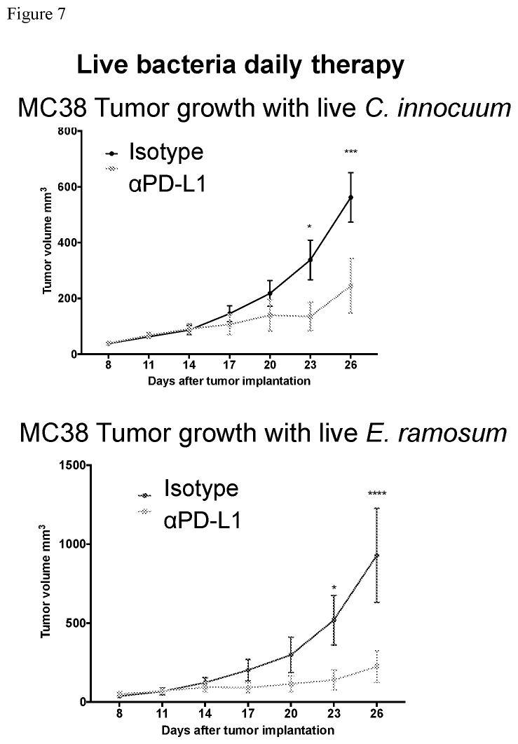

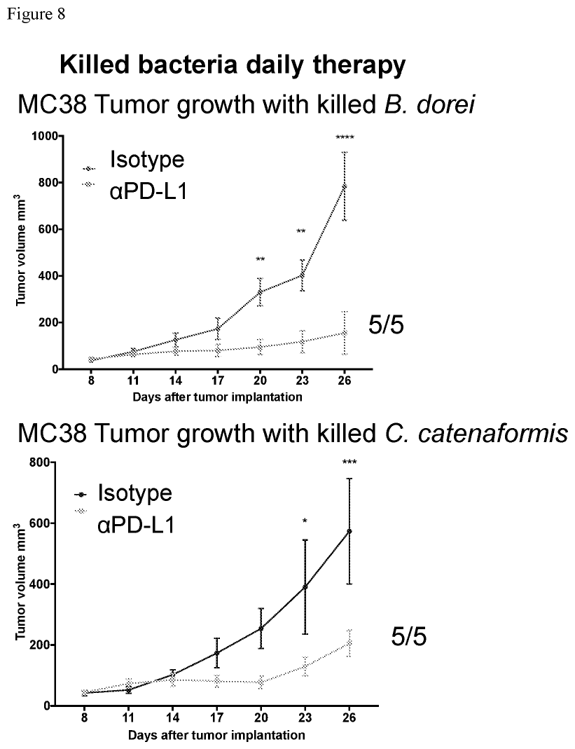

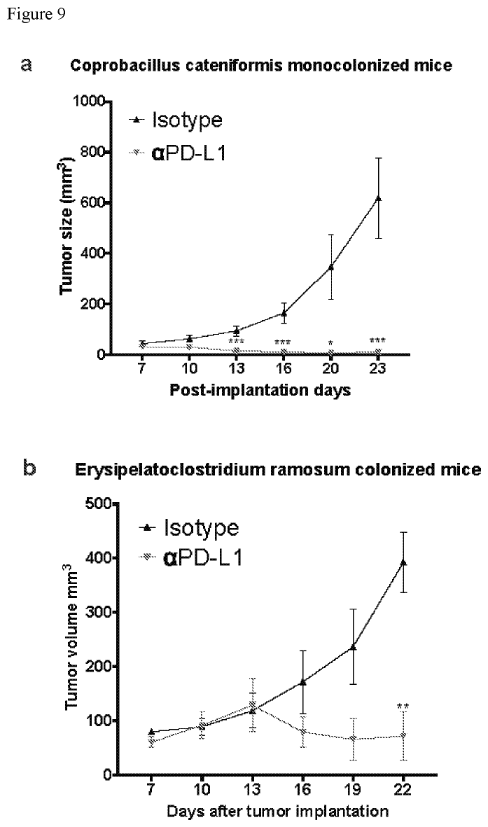

To test whether those bacteria indeed promote anti-tumor immunity, germ-free mice were colonized with a mixture of these six Clostridiales and one Bacteroides. Interestingly, 60% of the mice colonized with the Clostridiales/Bacteroides mix, and none of the GF mice, cleared the tumor after αPD-L1 treatment (FIG. 3a), indicating that this mix is effective to promote αPD-L1 dependent tumor clearance. To further narrow down the bacterial species that are needed for tumor clearance, GF mice were colonized with four of the seven Clostridiales/Bacteroides isolates and observed that these four also promote tumor clearance compared to GF mice (FIG. 3b). As seen by the data shown herein, several species alone promotes αPD-L1 dependent tumor clearance (FIG. 3c).

Colonization of a mix of bacteria disclosed herein all can induce effective anti-tumor immunity upon αPD-L1 treatment. The effect of the identified Clostridiales/Bacteroides was synergistic with αPD-L1 treatment because the Clostridiales themselves did not generate anti-tumor immunity. These bacterial strains could be used individually or in combination as a probiotic that promotes anti-tumor response to immunotherapy in cancer patients. Thus using the specific gut microbes in combination of the immune checkpoint blockade immunotherapy, would benefit a broader range of cancer patients who poorly respond to the current immunotherapy.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating or preventing cancer in a subject, the method comprising conjointly administering to a subject a composition comprising a Clostridia bacterium and an immunotherapeutic agent.

2. The method of claim 1, wherein at least 25% of the bacteria in the composition are a Clostridia bacterium.

3. The method of claim 1 or 2, wherein at least 50% of the bacteria in the composition are a Clostridia bacterium.

4. The method of any one of claims 1 to 3, wherein at least 75% of the bacteria in the composition are a Clostridia bacterium.

5. The method of any one of claims 1 to 4, wherein 100% of the bacteria in the composition are a Clostridia bacterium.

6. A method of treating or preventing cancer in a subject, the method comprising conjointly administering to a subject a composition comprising at least one bacteria species selected from Clostridium innocuum, Erysipelatoclostridium ramosum, Coprobacillus catenaformis, Bacteroides dorei, Flavonifractor plautii, and Blautia hydrogenotrophica and an immunotherapeutic agent.

7. The method of claim 6, wherein the composition comprises Erysipelatoclostridium ramosum.

8. The method of claim 7, wherein at least 25% of the bacteria in the composition are Erysipelatoclostridium ramosum bacteria.

9. The method of claim 7 or 8, wherein at least 50% of the bacteria in the composition are Erysipelatoclostridium ramosum bacteria.

10. The method of any one of claims 7 to 9, wherein at least 75% of the bacteria in the composition are Erysipelatoclostridium ramosum bacteria.

11. The method of any one of claims 7 to 10, wherein 100% of the bacteria in the composition are Erysipelatoclostridium ramosum bacteria.

12. The method of claim 6, wherein the composition comprises Coprobacillus catenaformis.

13. The method of claim 12, wherein at least 25% of the bacteria in the composition are Coprobacillus catenaformis bacteria.

14. The method of claim 12 or 13, wherein at least 50% of the bacteria in the composition are Coprobacillus catenaformis bacteria.

15. The method of any one of claims 12 to 14, wherein at least 75% of the bacteria in the composition are Coprobacillus catenaformis bacteria.

16. The method of any one of claims 12 to 15, wherein 100% of the bacteria in the composition are Coprobacillus catenaformis bacteria.

17. The method of claim 6, wherein the composition comprises Clostridium innocuum bacteria.

18. The method of claim 17, wherein at least 25% of the bacteria in the composition are Clostridium innocuum bacteria.

19. The method of claim 17 or 18, wherein at least 50% of the bacteria in the composition are Clostridium innocuum bacteria.

20. The method of any one of claims 17 to 19, wherein at least 75% of the bacteria in the composition are Clostridium innocuum bacteria.

21. The method of any one of claims 17 to 20, wherein 100% of the bacteria in the composition are Clostridium innocuum bacteria.

22. The method of claim 6, wherein the composition comprises Bacteroides dorei bacteria.

23. The method of claim 22, wherein at least 25% of the bacteria in the composition are Bacteroides dorei bacteria.

24. The method of claim 22 or 23, wherein at least 50% of the bacteria in the composition are Bacteroides dorei bacteria.

25. The method of any one of claims 22 to 24, wherein at least 75% of the bacteria in the composition are Bacteroides dorei bacteria.

26. The method of any one of claims 22 to 25, wherein 100% of the bacteria in the composition are Bacteroides dorei bacteria.

27. The method of claim 6, wherein the composition comprises Flavonifractor plautii bacteria.

28. The method of claim 27, wherein at least 25% of the bacteria in the composition are Flavonifractor plautii bacteria.

29. The method of claim 27 or 28, wherein at least 50% of the bacteria in the composition are Flavonifractor plautii bacteria.

30. The method of any one of claims 27 to 29, wherein at least 75% of the bacteria in the composition are Flavonifractor plautii bacteria.

31. The method of any one of claims 27 to 30, wherein 100% of the bacteria in the composition are Flavonifractor plautii bacteria.

32. The method of claim 6, wherein the composition comprises Blautia hydrogenotrophica bacteria.

33. The method of claim 32, wherein at least 25% of the bacteria in the composition are Blautia hydrogenotrophica bacteria.

34. The method of claim 32 or 33, wherein at least 50% of the bacteria in the composition are Blautia hydrogenotrophica bacteria.

35. The method of any one of claims 32 to 34, wherein at least 75% of the bacteria in the composition are Blautia hydrogenotrophica bacteria.

36. The method of any one of claims 32 to 35, wherein 100% of the bacteria in the composition are Blautia hydrogenotrophica bacteria.

37. A method of treating or preventing cancer in a subject, the method comprising conjointly administering to a subject a composition comprising Clostridium innocuum, Erysipelatoclostridium ramosum, Coprobacillus catenaformis, Bacteroides dorei, Flavonifractor plautii, and Blautia hydrogenotrophica, and an immunotherapeutic agent.

38. A method of treating or preventing cancer in a subject, the method comprising conjointly administering to a subject a composition comprising Clostridium innocuum, Erysipelatoclostridium ramosum, Coprobacillus catenaformis, and Bacteroides dorei, and an immunotherapeutic agent.

39. The method of any one of claims 1 to 38, wherein the composition enhances the anti-cancer activity of the immunotherapeutic agent.

40. The method of any one of claims 1 to 39, wherein the composition further comprises a pharmaceutically acceptable carrier.

41. The method of any one of claims 1 to 39, wherein the composition is a food product supplemented with the bacteria.

42. The method of claim 41, wherein the food product comprises a dairy product.

43. The method of claim 41, wherein the food product is a non-dairy food product.

44. The method of any one of claims 1 to 43, wherein the immunogenic composition comprises an immune checkpoint inhibitor.

45. The method of claim 44, wherein the immune checkpoint inhibitor comprises an antibody specific for an immune checkpoint protein selected from CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, PVRIG, (CD112R), HHLA2, butyrophilins, and A2aR.

46. The method of claim 44, wherein the immune checkpoint inhibitor is cemiplimab, nivolumab, pembrolizumab, atezolizumab, durvalumab, avelumab, ipilimumab and combinations thereof.

47. The method of any one of claims 1 to 46, wherein the subject had been administered an antibiotic prior to administration of the composition.

48. The method of claim 47, wherein the subject had been administered an antibiotic less than a week prior to administration of the composition.

49. The method of any one of claims 1 to 48, wherein the cancer is lung cancer, breast cancer, colon cancer, pancreatic cancer, renal cancer, stomach cancer, a GI cancer, liver cancer, bone cancer, hematological cancer, neural tissue cancer, melanoma, thyroid cancer, ovarian cancer, testicular cancer, prostate cancer, cervical cancer, vaginal cancer, or bladder cancer.

50. The method of any one of claims 1 to 49, wherein the cancer comprises a solid tumor.

51. The method of claim 50, wherein the tumor is an adenocarcinoma, an adrenal tumor, an anal tumor, a bile duct tumor, a bladder tumor, a bone tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngeal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor.

52. The method of claim 50 or 51, wherein the tumor is a primary tumor.

53. The method of claim 50 or 51, wherein the tumor is a metastatic tumor.

54. The method of any one of claims 1 to 53, wherein the subject has received a chemotherapy drug prior to administration of the agent.

55. The method of any one of claims 1 to 54, wherein the immunogenic composition is administered systemically.

56. The method of claim 55, wherein the immunogenic composition is administered intravenously.

57. The method of any one of claims 1 to 54, wherein the immunogenic composition is administered subcutaneously.

58. The method of any one of claims 1 to 54, wherein the immunogenic composition is administered intramuscularly.

59. The method of any one of claims 1 to 54, wherein the immunogenic composition is administered locally.

60. The method of any one of claims 1 to 54, wherein the immunogenic composition is administered orally.
